(19) United States
(12) Patent Application Publication
(10) Pub. No.: US 2021/0113381 A1
(43) Pub. Date: Apr. 22, 2021

(54) DARK FIELD REFLECTOMETRY

(71) Applicant: LUTRONIC VISION INC., Burlington, MA (US)

(72) Inventor: Seth Adrian MILLER, Longmont, CO (US)

(21) Appl. No.: 16/975,150

(22) PCT Filed: Mar. 6, 2018
(86) PCT No.: PCT/US18/20994

§ 371 Date: Aug. 24, 2020

(51) Int. Cl. A61F 9/008

(52) U.S. Cl. CPC A61F 9/00825; A61F 2009/00844

(57) ABSTRACT

Technologies are generally described to enhance a signal-to-noise ratio of reflectometry in laser treatment observation through capture of light scattered to the edge of the pupil and filtering of light scattered back around a center of the pupil. Thus, laser light reflected from the treatment site may be spatially filtered to remove a portion of the reflected laser light within a pre-selected angle from a normal of the eye. The captured laser light may be processed to observe an effect of the directed laser beam at the treatment site. The signal-to-noise ratio of the reflectometry may be increased through the spatial filtering.

### BACKGROUND

Unless otherwise indicated herein, the materials described in this section are not prior art to the claims in this application and are not admitted to be prior art by inclusion in this section.

Treatment of eye disease through laser application is a common approach. Tears or growths on the retina, and other diseases may be treated by application of laser beams (e.g., laser pulses) to the treatment site. To gauge an effect of the laser treatment, dosimetry may be performed using reflectometry, where the intensity of reflections of the laser pulse may be measured in real time. Laser beams generate heat at the treatment site, which in turn may result in formation of bubbles (through the expansion of fluids transforming into gases). Reflectometry may be able to detect bubbles because the large refractive index difference between bubbles and the surrounding fluid reflects light. Even though only a few bubbles may be created by the applied laser pulses, they may scatter light more effectively than the background, resulting in a measurable increase in the total reflectivity of the system. However, because the bubbles created in the system are typically small compared with a total spot size of the laser, the system signal-to-noise ratio may be poor resulting in less accurate measurements.

### SUMMARY

The present disclosure generally describes techniques to increase a signal-to-noise ratio of reflectometry in laser based eye treatment through capture of light scattered to the edge of the pupil and filtering of light scattered back from the pupil's center.

According to some examples, a method to increase a signal-to-noise ratio of reflectometry in laser based eye treatment is described. The method may include directing a laser beam to a treatment site within an eye through a pupil of the eye; spatially filtering reflected laser light from the treatment site to remove a portion of the reflected laser light within a pre-selected angle from a normal of the eye; and capturing the spatially filtered laser light, where the signal-to-noise ratio of the reflectometry is increased through the spatial filtering.

According to other examples, another method to increase a signal-to-noise ratio of reflectometry in laser based eye treatment is described. The method may include directing a laser beam to a treatment site within an eye through a pupil of the eye; capturing reflected laser light from the treatment site through a plurality of detectors; deriving a plurality of signals from the captured laser light by the plurality of detectors; and removing a portion of the derived plurality of signals that corresponds to a portion of the captured laser light within a pre-selected angle from a normal of the eye to increase the signal-to-noise ratio of the reflectometry.

According to further examples, a laser treatment system may include a laser configured to direct a laser beam to a treatment site within an eye through a pupil of the eye and a detection module coupled to the laser. The detection module may be configured to spatially filter reflected laser light from the treatment site to remove a portion of the reflected laser light within a pre-selected angle from a normal of the eye; and capture the spatially filtered laser light, where the signal-to-noise ratio of the reflectometry is increased through the spatial filtering.

According to some examples, another laser treatment system may include a laser configured to direct a laser beam to a treatment site within an eye through a pupil of the eye; a detection module coupled to the laser and comprising a plurality of detectors, the detection module configured to capture reflected laser light from the treatment site through the plurality of detectors; and a processing module coupled to the laser and the detection module. The processing module may be configured to derive a plurality of signals from the captured laser light by the plurality of detectors; and remove a portion of the derived plurality of signals that corresponds to a portion of the captured laser light within a pre-selected angle from a normal of the eye to increase the signal-to-noise ratio of the reflectometry.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates raw optical reflectometry data from an example laser treatment system;

FIG. 2 illustrates processed optical reflectometry data from an example laser treatment system;

FIG. 3 illustrates how reflectometry signal within a predefined angle may be captured by an annular detector;

FIG. 4 illustrates major functional blocks in an example laser treatment system with enhanced signal-to-noise ratio of reflectometry through capture of light scattered to the edge of the pupil and filtering of light scattered back from the pupil's center;

FIG. 5 illustrates an example laser treatment system where a signal-to-noise ratio of reflectometry in laser based eye treatment through capture of light scattered to the edge of the pupil and filtering of light scattered back from the pupil's center;

FIG. 6 illustrates how light encountering a refractive index change may partially pass, and partially reflect back towards the source;

FIG. 7 illustrates an angular distribution of light scattered off 1 μm bubbles in a water medium;

FIG. 8 illustrates a plot of intensity of light scattered off 1 μm bubbles by Mie scattering, overlaid with the footprint of directly reflected laser light;

FIG. 9 illustrates a conceptual diagram of an example implementation, where a modified dark field capture system modelled after a spot ring bicentric condenser may be used to enhance signal-to-noise ratio of reflectometry through capture of light scattered to the edge of the pupil and filtering of light scattered back from the pupil's center;

FIG. 10 illustrates a computing device, which may be used to enhance signal-to-noise ratio of reflectometry through capture of light scattered to the edge of the pupil and filtering of light scattered back from the pupil's center;

FIG. 11 is a flow diagram illustrating an example method to enhance signal-to-noise ratio of reflectometry through capture of light scattered to the edge of the pupil and filtering of light scattered back from the pupil's center that may be performed by a computing device such as the computing device in FIG. 10; and

FIG. 12 illustrates a block diagram of an example computer program product, all arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and/or computer program products related to increase of a signal-to-noise ratio of reflectometry in laser based eye treatment through capture of light scattered to the edge of the pupil and filtering of light scattered back from the pupil's center.

Briefly stated, technologies are generally described to enhance a signal-to-noise ratio of reflectometry in laser treatment observation through capture of light scattered to the edge of the pupil and filtering of light scattered back around a center of the pupil. Thus, laser light reflected from the treatment site may be spatially filtered to remove a portion of the reflected laser light within a pre-selected angle from a normal of the eye. The captured laser light may be processed to observe an effect of the directed laser beam at the treatment site. The signal-to-noise ratio of the reflectometry may be increased through the spatial filtering.

In the following figures and diagrams, the positioning, structure, and configuration of example systems, devices, and implementation environments have been simplified for clarity. Embodiments are not limited to the configurations illustrated in the following figures and diagrams. Furthermore, examples are provided herein for treatment of diseases on retinal pigment epithelium (RPE) layer. Embodiments are not limited to enhancement of signal-to-noise ratio of reflectometry in laser treatment observation for RPE treatments, and may be implemented in conjunction with laser treatments of other ophthalmologic conditions as well.

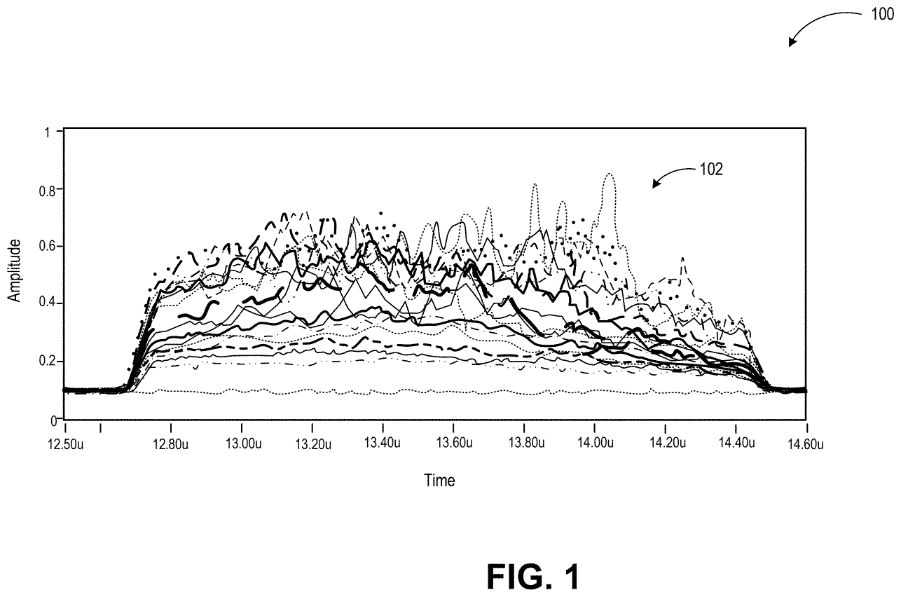

FIG. 1 illustrates raw optical reflectometry data from an example laser treatment system.

As discussed previously, laser beams generate heat at the treatment site, which in turn may result in formation of bubbles (through the expansion of fluids transforming into gases) on the retina in eye disease treatments through a laser beam application. Reflectometry generally refers to techniques for use of the reflection of waves at surfaces and interfaces to detect or characterize objects. In ophthalmology, reflectometry is used as a dosimetry approach to monitor laser surgery progress by detecting backscattered light from a treatment site (e.g., surface of the retina) to which a laser beam is applied for surgical treatment purposes. Reflectometry may be able to detect bubbles because the large refractive index difference between bubbles and the surrounding fluid reflects light. Even though only a few bubbles may be created by the applied laser pulses, they may scatter light more effectively than the background, resulting in a measurable increase in the total reflectivity of the system. However, because the bubbles created in the system are typically small compared with a total spot size of the laser, the system signal-to-noise ratio may be poor resulting in less accurate measurements.

Diagram 100 shows an example raw reflectometry signal 102, where a strong response may be only five times the intensity of the baseline light reflecting from the unheated RPE layer. The challenge may be made worse by a refractive index difference between the heated (e.g., heating of melanosomes through the laser beam) and body temperature regions of the fluid in the treatment. The “unheated” or body temperature regions may also reflect light back to a detector. In the example time-domain reflectometry signal, the increase in the amplitude at the beginning of signal collection may be due to heating. In practical implementations, several hundred nanoseconds may pass before a bubble nucleates. As a result, a spike in the intensity due to bubble formation may increase the signal amplitude about 30% or less from the thermal baseline.

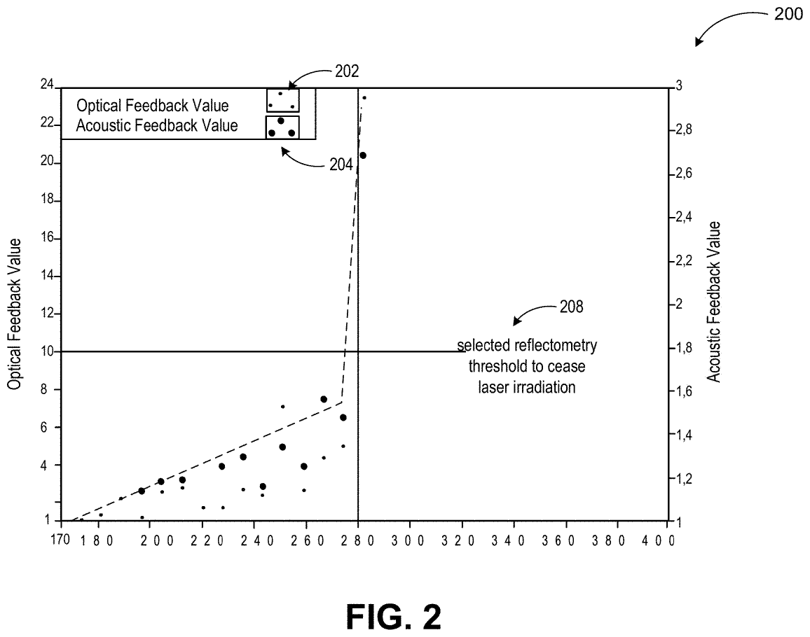

FIG. 2 illustrates processed optical reflectometry data from an example laser treatment system.

Acoustic feedback (detection of bubble formation through pressure waves) is a conventionally used approach in observing an effect of laser eye treatments, but that approach may be inaccurate and cumbersome in practical implementation scenarios. Optical feedback (reflectometry) is promising compared to acoustic feedback due to less complex equipment set up (no requirement for a probe to touch a surface of the eye, for example) and potential advantages in accuracy. However, standard reflectometry results may not provide substantially improved results over acoustic feedback.

Between both ‘structural’ and ‘thermal’ reflections, the challenge for accurate and useful observation of laser treatment results may be to extract a bubble signature from the background, so that even in the processed data, the difference in signal strength between a “detect” and “ignore” event (for example in a treatment continuation estimator) may be exceedingly small (close or at a noise level of the total signal), as shown in diagram 200.

The processed optical data in diagram 200 includes optical feedback values 202 and acoustic feedback values 204. In the example results, a sub-threshold event may have an acoustic feedback value of 7, while the reflectometry threshold is 10. Thus, a selected reflectometry threshold to cease laser treatment 208 (based on the feedback) may have a small margin for distinguishing success vs. failure of the treatment.

On the other hand, a system to enhance a signal-to-noise ratio of reflectometry in laser treatment observation through capture of light scattered to the edge of the pupil and filtering of light scattered back around a center of the pupil may enhance safety and overall efficacy of the treatment by separating the optical signal due to bubble formation from a background noise due to RPE and thermal reflections. Thus, the pupil may be used to define an outer angular range of collected light.

In practical implementations, greater annular range of collected light free from reflection may provide higher accuracy in observation through enhancement of the signal-to-noise ratio. The annular range may be practically limited by pupil width, for example, 7°. However, different individuals may have pupils with an angular range smaller than 7°, which may decrease the amount of light that can be collected by an external detector, and thus decrease the signal-to-noise ratio. Selecting the inner spatial filtering range less than 2° may result in higher likelihood of capturing direct reflections, which may again decrease the signal-to-noise ratio. Moreover, abnormal structures such as white regions on the RPE may exist in some eyes and scatter the direct reflections in unexpected directions. Such structures may also cause a decrease the signal-to-noise ratio. In a practical scenario, up to ten-fold increase in the signal-to-noise ratio may be achieved employing some embodiments, but that increase may be subject to the above-discussed degradations based on individual characteristics.

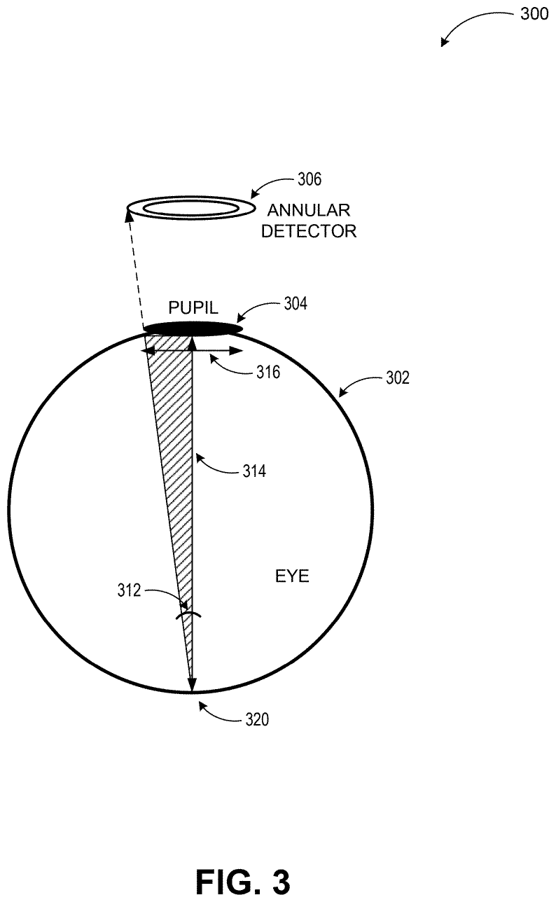

FIG. 3 illustrates how reflectometry signal within a predefined angle may be captured by an annular detector, arranged in accordance with at least some embodiments described herein.

Diagram 300 shows a simplified illustration of an eye 302 undergoing laser treatment, where spatial filtering may enhance reflectometry results. If the eye's diameter 314 is approximated by about 24 mm and the pupil's (304) diameter 316 is approximated by about 4 to 8 mm, reflected laser light (used for reflectometry) may exit the eye 302 at an angle range of about 5 to 9 degrees (312). The approximation of the pupil's diameter 316 include dilation of the pupil due to dark environment or mydriasis. The excitation of the radial fibers of the iris which increases the pupillary aperture is referred to as a mydriasis. Mydriasis may be caused by disease, trauma, or drugs (e.g., medication delivered during surgery). Because a dilated pupil may allow exit of light at the higher angles (e.g., a 4-8 mm pupil in a 24 mm deep eye may allow light to exit at angles of 5 to 9 degrees), scattered light that does not contain thermal reflections may be captured outside of that range. Thus, light outside of the range may be omitted since is it likely to include background noise only.

For an approximate eye diameter 314 of about 24 mm, reflected laser light may bounce off the thermal front and exit the eye 302 within an approximate angle of about ±1.2° from the incident line of the surgical laser (normal of the eye 302). Bubbles may scatter light across a wide distribution of angles, but reflectometry implementations capture light reflected at a single angle, directly back from the treatment site 320 along the path of the laser (0° from normal). By (spatially) filtering light that is scattered at angles >1.2°, a majority of the light scattered directly from the back of the eye or from thermals may be filtered out, while still collecting light scattered from the bubbles. In some embodiments, an annular detector 306 may be used to collect the reflected laser light (and filter out the center portion) thereby reducing a system background noise substantially and allowing enhanced detection of bubbles. In other embodiments, the spatial filtering range (1.2°) may be selected differently depending on an actual diameter of the eye, pupil size, laser wavelength, expected bubble sizes, etc. The 1.2° spatial filtering angle used as example herein is an estimate based on the standard eye model of scattering and a 100 μm laser spot diameter. For narrower spot sizes, the reflections may become more tightly focused and the spatial filtering angle may be selected smaller. By contrast, if the optical structure of the eye causes a reflected signal to diverge slightly on exit, the spot size for reflection may be effectively larger, and the spatial filtering angle may be increased. For example, a 200 μm laser spot diameter may be implemented in some systems. In the latter cases, 1.2° may be taken as a minimum. Blocking of light at less than 1.2°, 1.5°, or 2° may be practically preferred based patient variability.

In some examples, the annular detector 306 may be configured with a central blocking component to filter out the reflected light within the predefined angle. The central blocking component may be held in place through a number of spokes or comparable mechanisms. For example, a black or mirrored dot may be painted at an appropriate location on the detector or a lens in front of it. In other examples, curved mirrors may be used to collect only annular light and reflect the collected light back to a central collecting optics. Combination of lenses that are formed so that light in the center diverges rather than converges may also be used to implement embodiments. In some examples, the filtered region may be adjustable, which may be accomplished through an adjustable mechanical element (e.g., the blocking component) or electronically (e.g., by computationally filtering the central regions of a multi-pixel detector).

A resulting reflectometry from the use of an example filtered collection configuration may have a lower baseline noise level with far less of a response to pulses that do not create bubbles. Thus, a signal-to-noise ratio of the captured laser light (reflected laser light of interest over background reflections) may be enhanced allowing detection of smaller bubbles, and thus provide an ophthalmologist with finer control over the surgical procedure, and increased confidence in the safety and efficacy of the treatment.

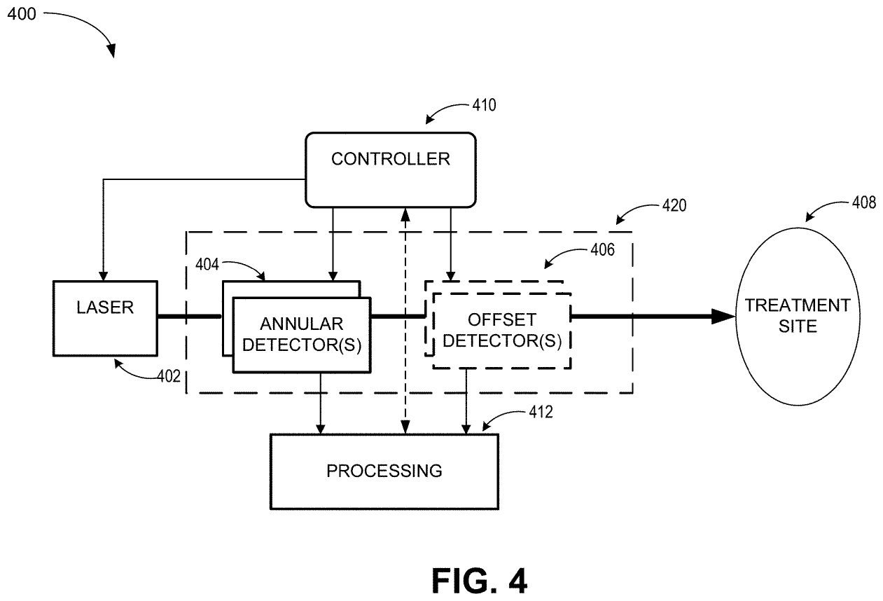

FIG. 4 illustrates major functional blocks in an example laser treatment system with enhanced signal-to-noise ratio of reflectometry through capture of light scattered to the edge of the pupil and filtering of light scattered back from the pupil's center, arranged in accordance with at least some embodiments described herein.

An example laser treatment system as shown in diagram 400 may include a laser 402 configured to direct a laser beam to a treatment site 408 within an eye through a pupil of the eye and a detection module 420 coupled to the laser 402. The detection module 420 may be configured to spatially filter reflected laser light from the treatment site 408 to remove a portion of the reflected laser light within a pre-selected angle from a normal of the eye and capture the spatially filtered laser light to enhance a signal-to-noise ratio of the reflectometry. The detection module 420 may include annular detector(s) 404 or offset detector(s) 406, for example. The spatial filtering may be accomplished through placing a single detector at an angle offset from the line of the laser, placing a series of detectors in an annular (or partial annular) configuration around the line of the laser, or using a single detector that captures all light escaping from the pupil, but placing an aperture in the center in some examples.

The pre-selected angle from the normal of the eye may be determined based on a diameter of the laser beam. For example, the portion of the reflected laser light within about 1.2 degrees from the normal of the eye may be removed for a laser beam of about 100 μm diameter. In other examples, another portion of the reflected laser light outside of about 9 degrees from the normal of the eye may be removed. The example laser treatment system may also include a processing module 412 coupled to the laser 402 and the detection module 420. The processing module 412 may be configured to process the captured laser light to observe an effect of the directed laser beam at the treatment site 408. The system may also include a controller 410 coupled to the laser 402, the detection module 420, and the processing module 412. The controller 410 may be configured to control and coordinate operations of the laser 402, the detection module 420, and the processing module 412.

Another laser treatment system according to examples may include a detection module comprising a plurality of detectors and configured to capture reflected laser light from the treatment site through the plurality of detectors. The processing module coupled to the laser and the detection module may be configured to derive a plurality of signals from the captured laser light by the plurality of detectors and remove a portion of the derived plurality of signals that corresponds to a portion of the captured laser light within a pre-selected angle from a normal of the eye to increase the signal-to-noise ratio of the reflectometry. The processing module may then process a remaining portion of the plurality of signals to observe an effect of the directed laser beam at the treatment site. Thus, reflected light may be captured over a broad area using a detector array, for example, and digitally removed.

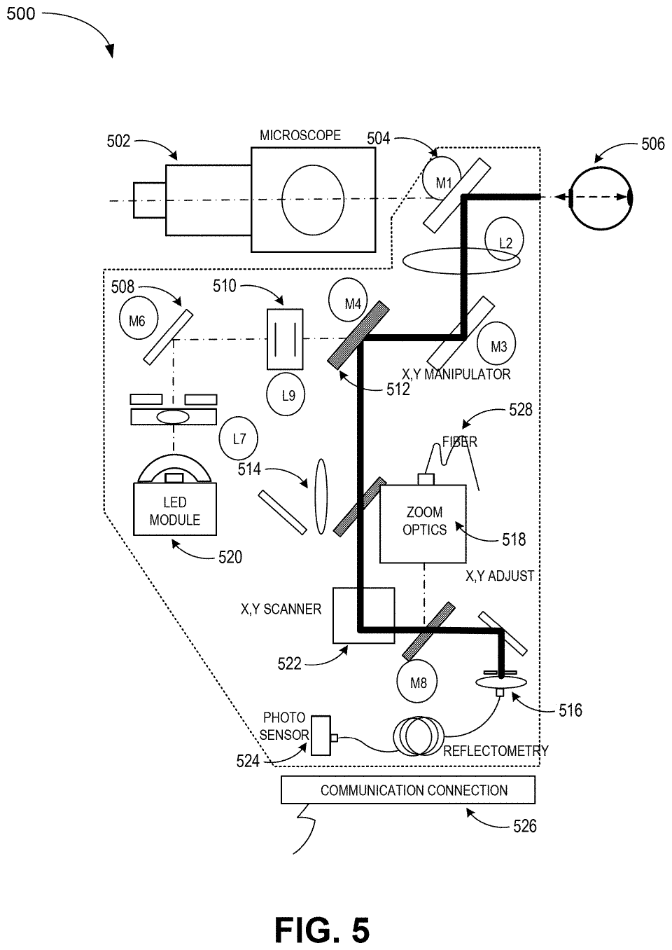

FIG. 5 illustrates an example laser treatment system where a signal-to-noise ratio of reflectometry in laser based eye treatment through capture of light scattered to the edge of the pupil and filtering of light scattered back from the pupil's center, arranged in accordance with at least some embodiments described herein.

Some embodiments are directed to increase of signal-to-noise ratio in laser reflectometry to incorporate dark-field imaging through capture of light reflected at high angles (e.g., >±1.2° from the normal plane of the eye) while fully or partially excluding light from lower angles (e.g., <±1.2° from the normal plane). The signal-to-noise ratio of the reflectometry may be substantially increased by filtering out photons reflected by the thermal front, while capturing the light reflected from the bubbles formed through the heating action by the directed laser beam. Light reflected from the treatment site (e.g., a location on the RPE) contains photons reflected by both bubbles—the desired signal—and the thermal wave front created by laser heating. As discussed herein, directed laser light may bounce off the thermal front and exit the eye within an angle of about ±1.2° from the incident line of the surgical laser (normal plane of the eye). However, bubbles scatter light to significantly higher angles. Because a dilated pupil may allow exit of light at the higher angles (e.g., a 4-8 mm pupil in a 24 mm deep eye may allow light to exit at angles of 5°-9°), scattered light that does not contain thermal reflections may be captured. For example, light collected at angles from 1.2°-9° may be primarily light scattered from bubbles, not thermals. By capturing this “dark field” light, the desired signal (from the bubbles) may be separated from the background noise (from thermals), thereby substantially increasing the signal-to-noise ratio and/or dosimetry.

Diagram 500 shows an example laser treatment system with selected components. A laser treatment system according to embodiments may include additional or fewer components and may be formed using other configurations. An example configuration may include laser source. The laser source may include a semiconductor laser diode, a super-luminescent laser diode, a chemical laser, a gas laser, a solid-state laser, or an optical fiber diode, for example. The laser beam from the laser source (e.g., zoom optics module 518) may be delivered to the treatment site 506 through the fiber 528. Various optical components such as lenses 510 and 514, and mirrors 508, 512, and 504 may be used to focus, direct, and otherwise manipulate the reflected laser beam from the treatment site 506. Some of the mirrors (and lenses) may be manipulators that through mechanical or electronic action redirect the laser light. Other mirrors and lenses may be used to split the laser light (directed and/or reflected) such that the reflected laser light can be observed by devices such as microscope 502. For example, transparent or semi-transparent mirrors may be used to provide splitting of reflected light.

Light reflected from the treatment site 506 may also be directed to a filter module 516, which may spatially or electronically filter out a central portion of the reflected light as discussed herein. The filtered light may then be provided to a detector (e.g., photo sensor) 524 for processing. The detector 524 may be a photodiode, an active-pixel sensor (APS), a Cadmium Zinc Telluride radiation detector, a charge-coupled device (CCD), a Mercury Cadmium Telluride detector, a reverse-biased light emitting diode (LED), a photoresistor, a phototransistor, or a quantum dot photoconductor, photomultiplier tube (PMT), for example. A communication connection 526 may provide the signal(s) from the detector 524 to other devices such as a controller to be displayed to a health professional controlling the laser or to be used in automatic dosimetry decisions (whether to cease the laser treatment or continue). Additional components such as zoom optics 518 or x-y scanner 522 may provide further control of the process and allow an operator to focus on the treatment area and observe an effect of the directed laser beam.

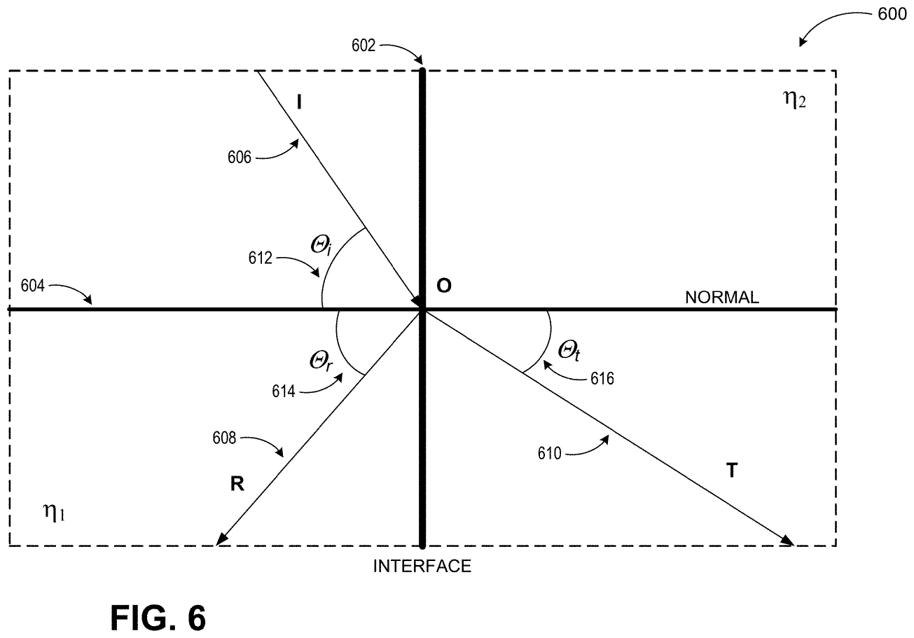

FIG. 6 illustrates how light encountering a refractive index change may partially pass, and partially reflect back towards the source, arranged with at least some embodiments described herein.

A directed laser beam heating of the treatment site (e.g., a melanosome) during surgical procedure may create a thermal front that expands from the treatment site and travels towards the front of the eye at approximately the speed of sound in the fluid. The speed of sound in water is about 1500 m/s; in other words, the thermal front may travel approximately one millimeter in 1 μs. At this distance, the fine structure associated with the melanosomes (approximately 10 μm in size) may have dissipated, and the thermal wave front may become, for optical purposes, a straight line.

The straight line of the thermal front may reflect light classically, similar to a mirror, as described by the Fresnel equations. As described graphically in diagram 600, incident light I (606) impinging on an interface 602 at an angle of θᵢ 612 may be reflected back as R 608 at an angle of θᵣ 614, where θᵣ=−θᵢ relative to the normal 604 of the interface plane. A portion of the incident light I 606 may be transmitted through as T 610 at an angle of θₜ 616.

In a surgical laser treatment system, the directed laser beam may be incident at 0° (the normal 604 itself). Thus, thermal front may reflect the incident light back at 0°. An example surgical laser beam may have a 100 μm diameter spot. Such a laser beam, returning through an eye that is approximately 24 mm deep, may consume a 360°*0.1/(3.14*24*2)=2.4° of arc, or about 1.2° of arc on either side of the normal. In other words, light reflected by the thermal front may be confined to about ±1.2° away from the normal.

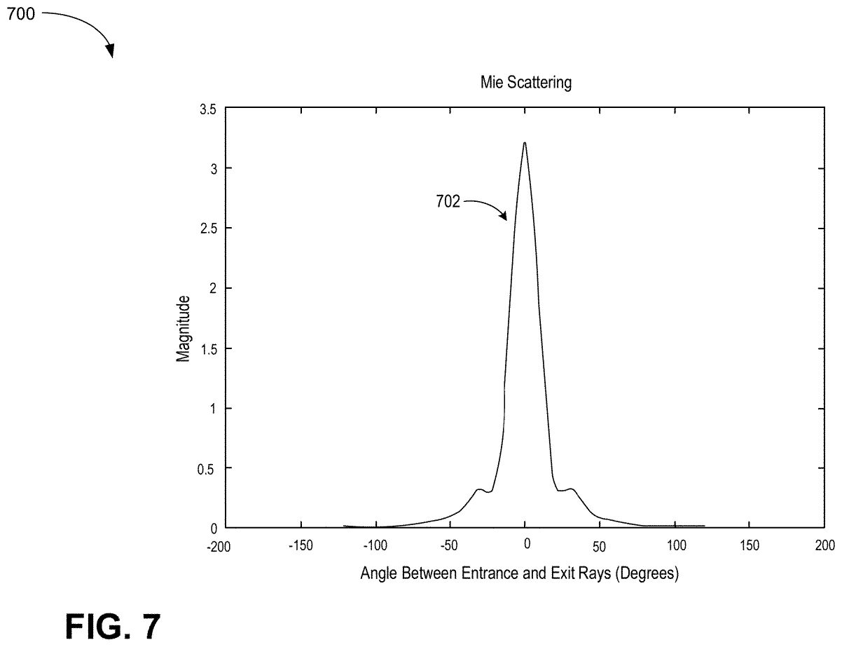

FIG. 7 illustrates angular distribution of light scattered off 1 μm bubbles in a water medium, arranged with at least some embodiments described herein.

The reflection of light interacting with bubbles may be described more accurately by Mie scattering, rather than the Fresnel equations. Mie scattering is the dominant mechanism for light scattered by particles (except those much smaller than the wavelength of light), and may reflect light not back to the normal, but across a wide range of angles up to about 90°.

Diagram 700 shows a model of Mie scattering 702 in a system containing 1 μm bubbles of refractive index 1 in a medium of refractive index of 1.5, where the horizontal axis represents an angle between an entrance and an exit of the laser light (directed and reflected beams). As can be seen in the diagram, a greatest intensity of reflection may be at θ=0°, and a majority of light scattered into other angles. In a practical example, Mie scattered light may be still about ¾ the intensity as normally reflected light at an angle of 7°. Yet, the area for light capture may be far larger than a single laser spot. As a result, an annular detector, configured to capture the light reflected from bubbles at ±5-7° (a 2° arc), may actually have >10× the intensity of a detector configured to capture light from ±1° (also 2° of arc) around the normal axis. Meanwhile, light reflected from thermals, which are the primary contributor to noise, may be nearly non-existent at the higher angles (e.g., ±5-7°).

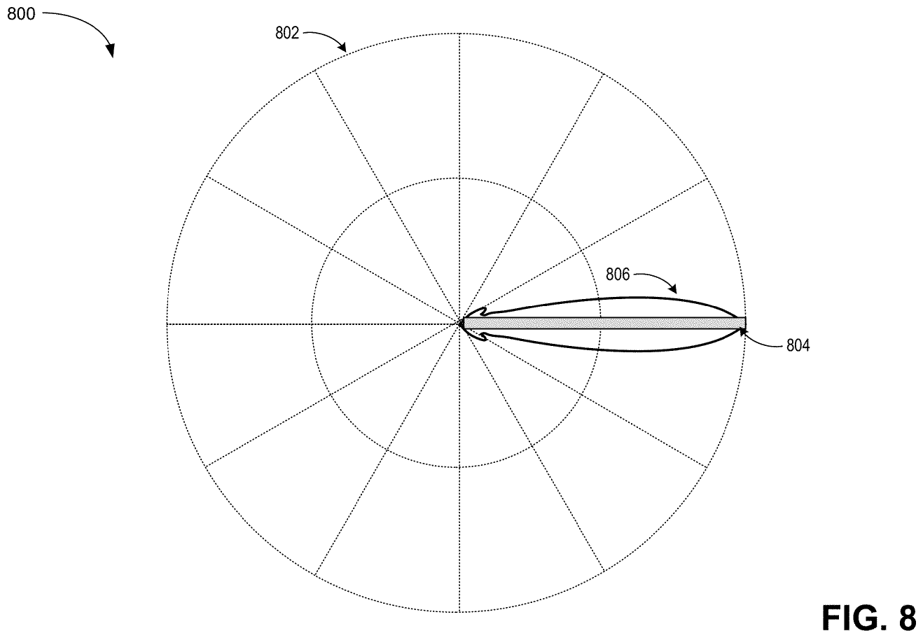

FIG. 8 illustrates a plot of intensity of light scattered off 1 μm bubbles by Mie scattering, overlaid with the footprint of directly reflected laser light, arranged with at least some embodiments described herein.

Diagram 800 includes a polar plot 802 that shows a representation of the directed laser beam 804 overlaid with the intensity of light 806 as a function of angle traced in polar coordinates. As can be seen, a majority of the intensity of the scattered light may be outside of the directly reflected path of the laser beam.

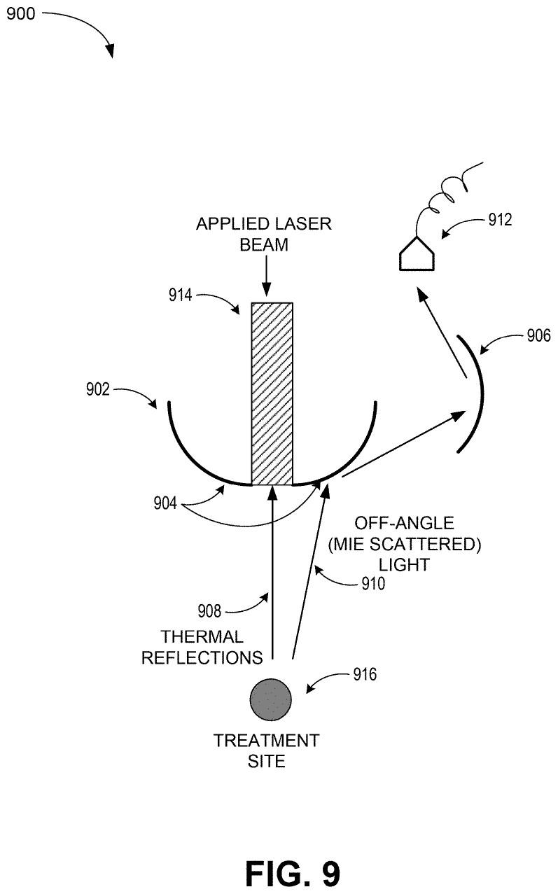

FIG. 9 illustrates a conceptual diagram of an example implementation, where a modified dark field capture system modelled after a spot ring bicentric condenser may be used to enhance signal-to-noise ratio of reflectometry through capture of light scattered to the edge of the pupil and filtering of light scattered back from the pupil's center, arranged with at least some embodiments described herein.

In an example embodiment, shown in diagram 900, a dark field capture system may be modelled after a spot ring bicentric condenser, for example. The incident laser beam 914 may pass through a partially mirrored (904) lens 902. The transparent middle portion of the lens 902 may pass the applied laser beam through to the treatment site 916 (e.g., the eye). In response to the applied laser beam 914, thermal reflections 908 and off-angle (reflected) light 910 may arrive at the lens 902 from the treatment site 916. The mirrors 904 of the lens 902 may reflect the off-angle light 910 outward to a second mirror 906, which in turn may focus the off-angle light 910 to a detector such as a photomultiplier tube (PMT) 912. Thus, the partially mirrored lens 902 and second mirror 906 combination may provide spatial filtering for the reflected light as discussed herein. In another configuration, the lens may be flattened, so no focusing of the laser takes place. Alternatively, the lens may be drilled out centrally, so the laser beam passes through free space. Other configurations may also be implemented using the principles described herein, where reflected light outside of a pre-selected angle (e.g., >1.2°) is captured and reflected light within the pre-selected angle is partially or wholly excluded from capture.

Practically, a detector configured to collect light at >1.2° from normal may deliver higher signal-to-noise ratio (higher scattering signal vs thermal signal) than a conventional detector. It should be noted that alignment need not be perfect: any system that removes some light from <1.2° and enhances light at >1.2° may deliver a signal-to-noise ratio improvement over conventional approaches. All of the reflected light (within the filtering range) need not be captured. Because the area of the annulus is typically much larger than the area of the laser spot, even if only part of the annulus is collected, overall signal strength may still be substantially higher than if only directly reflected light is captured.

In other example embodiments, the detector may be set to collect light at >2° from the normal, in order to provide extra margin to ensure that normally reflected light is excluded, for example. In further example embodiments, reflected light >3° may be collected, etc. Using a larger detector in an annular configuration may both increase total signal, and decrease the background noise level. Because a system according to embodiments is configured to detect bubbles, which scatter, while excluding reflections from thermals (which do not scatter), dark field collection may substantially enhance signal-to-noise ratio of the system. This may allow detection of smaller bubbles, and provide higher confidence in thresholding decisions. A signal-to-noise ratio as used herein refers to a ratio of desired signal (laser light reflected from the bubbles) to a background noise (light generated by thermals and environment).

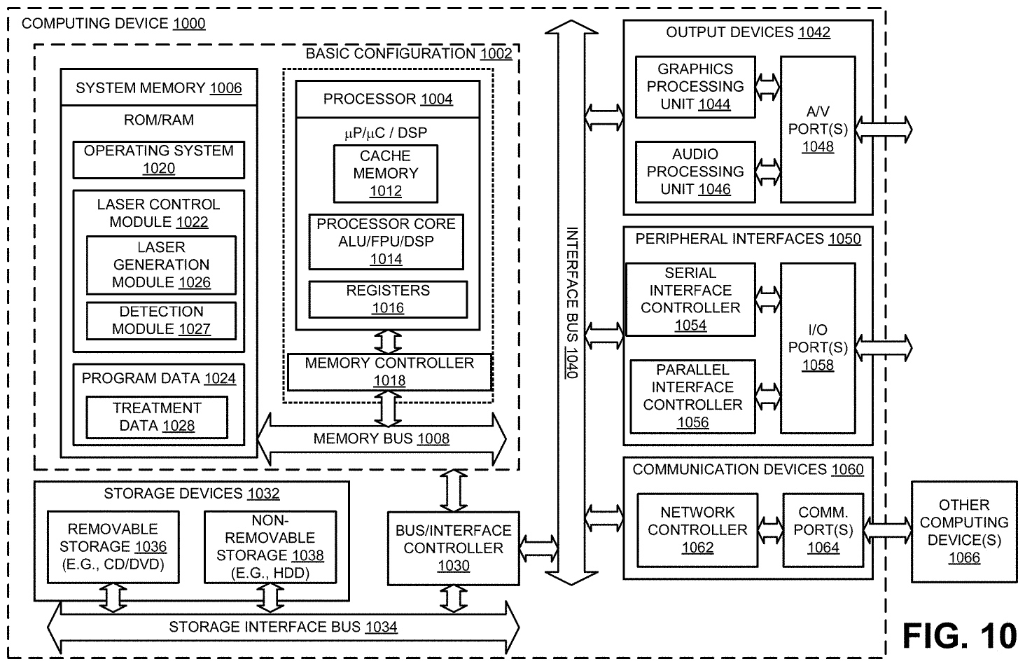

FIG. 10 illustrates a computing device, which may be used to enhance signal-to-noise ratio of reflectometry through capture of light scattered to the edge of the pupil and filtering of light scattered back from the pupil's center, arranged with at least some embodiments described herein.

In an example basic configuration 1002, the computing device 1000 may include one or more processors 1004 and a system memory 1006. A memory bus 1008 may be used to communicate between the processor 1004 and the system memory 1006. The basic configuration 1002 is illustrated in FIG. 10 by those components within the inner dashed line.

Depending on the desired configuration, the processor 1004 may be of any type, including but not limited to a microprocessor (μP), a microcontroller (μC), a digital signal processor (DSP), or any combination thereof. The processor 1004 may include one or more levels of caching, such as a cache memory 1012, a processor core 1014, and registers 1016. The example processor core 1014 may include an arithmetic logic unit (ALU), a floating point unit (FPU), a digital signal processing core (DSP Core), or any combination thereof. An example memory controller 1018 may also be used with the processor 1004, or in some implementations the memory controller 1018 may be an internal part of the processor 1004.

Depending on the desired configuration, the system memory 1006 may be of any type including but not limited to volatile memory (such as RAM), non-volatile memory (such as ROM, flash memory, etc.) or any combination thereof. The system memory 1006 may include an operating system 1020, a laser control module 1022, and program data 1024. The laser control module 1022 may include a laser generation module 1026 and a detection module 1027. The laser generation module 1026 may direct a laser beam to a treatment site such as a location on the cornea of an eye through the pupil of the eye. The detection module 1027 may spatially filter reflected laser light from the treatment site to remove a portion of the reflected laser light within a pre-selected angle from a normal of the eye and capture the spatially filtered laser light to increase the signal-to-noise ratio of the reflectometry through the spatial filtering. The program data 1024 may include, among other data, treatment data 1028 or the like, as described herein.

The computing device 1000 may have additional features or functionality, and additional interfaces to facilitate communications between the basic configuration 1002 and any desired devices and interfaces. For example, a bus/interface controller 1030 may be used to facilitate communications between the basic configuration 1002 and one or more data storage devices 1032 via a storage interface bus 1034. The data storage devices 1032 may be one or more removable storage devices 1036, one or more non-removable storage devices 1038, or a combination thereof. Examples of the removable storage and the non-removable storage devices include magnetic disk devices such as flexible disk drives and hard-disk drives (HDDs), optical disk drives such as compact disc (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer readable instructions, data structures, program modules, or other data.

The system memory 1006, the removable storage devices 1036 and the non-removable storage devices 1038 are examples of computer storage media. Computer storage media includes, but is not limited to, RAM, ROM, EEPROM, flash memory or other memory technology, CD-ROM, digital versatile disks (DVDs), solid state drives, or other optical storage, magnetic cassettes, magnetic tape, magnetic disk storage or other magnetic storage devices, or any other medium which may be used to store the desired information and which may be accessed by the computing device 1000. Any such computer storage media may be part of the computing device 1000.

The computing device 1000 may also include an interface bus 1040 for facilitating communication from various interface devices (e.g., one or more output devices 1042, one or more peripheral interfaces 1050, and one or more communication devices 1060) to the basic configuration 1002 via the bus/interface contro11er1030. Some of the example output devices 1042 include a graphics processing unit 1044 and an audio processing unit 1046, which may be configured to communicate to various external devices such as a display or speakers via one or more A/V ports 1048. One or more example peripheral interfaces 1050 may include a serial interface controller 1054 or a parallel interface controller 1056, which may be configured to communicate with external devices such as input devices (e.g., keyboard, mouse, pen, voice input device, touch input device, etc.) or other peripheral devices (e.g., printer, scanner, etc.) via one or more I/O ports 1058. An example communication device 1060 includes a network controller 1062, which may be arranged to facilitate communications with one or more other computing devices 1066 over a network communication link via one or more communication ports 1064. The one or more other computing devices 1066 may include servers at a datacenter, customer equipment, and comparable devices.

The network communication link may be one example of a communication media. Communication media may be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

The computing device 1000 may be implemented as a part of a general purpose or specialized server, mainframe, or similar computer that includes any of the above functions. The computing device 1000 may also be implemented as a personal computer including both laptop computer and non-laptop computer configurations.

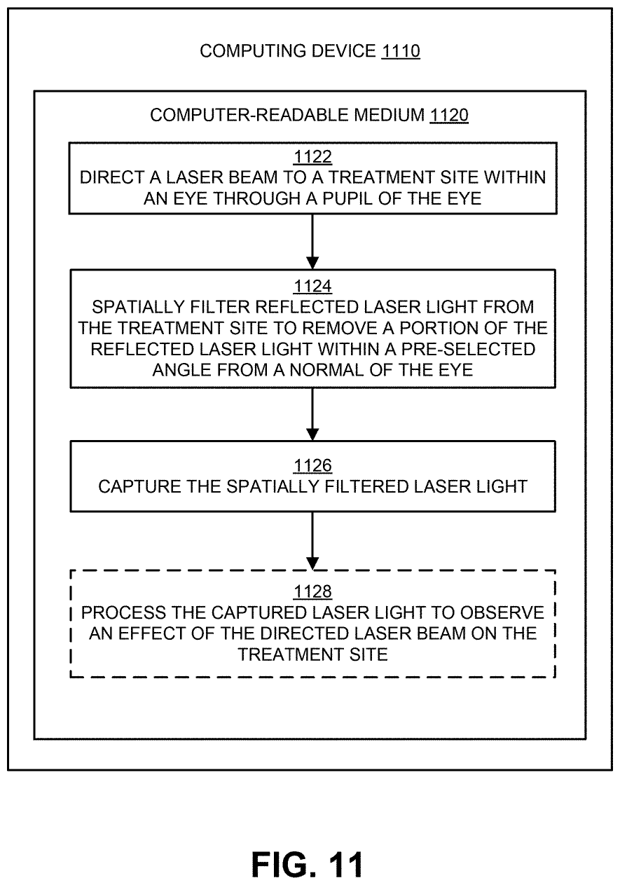

FIG. 11 is a flow diagram illustrating an example method to enhance signal-to-noise ratio of reflectometry through capture of light scattered to the edge of the pupil and filtering of light scattered back from the pupil's center that may be performed by a computing device such as the computing device in FIG. 10, arranged with at least some embodiments described herein.

Example methods may include one or more operations, functions or actions as illustrated by one or more of blocks 1122, 1124, 1126, and/or 1128, and may in some embodiments be performed by a computing device such as the computing device 1100 in FIG. 11. The operations described in the blocks 1122-1128 may also be stored as computer-executable instructions in a computer-readable medium such as a computer-readable medium 1120 of a computing device 1110.

An example process to enhance signal-to-noise ratio of reflectometry through capture of light scattered to the edge of the pupil and filtering of light scattered back from the pupil's center may begin with block 1122, “DIRECT A LASER BEAM TO A TREATMENT SI WITHIN AN EYE THROUGH A PUPIL OF THE EYE”, where a laser beam (e.g., successive laser pulses of select amplitude, pulse width, and frequency) may be directed to a treatment site such as a melanosome in the RPE cells of the eye.

Block 1122 may be followed by block 1124, “SPATIALLY FILTER REFLECTED LASER LIGHT FROM THE TREATMENT SITE TO REMOVE A PORTION OF THE REFLECTED LASER LIGHT WITHIN A PRE-SELECTED ANGLE FROM A NORMAL OF THE EYE”, where filtering may be applied to reflected laser light from the treatment site in order to capture dark field reflectometry and increase a signal-to-noise ratio of reflected light from the formed bubbles to background noise such as light generated by thermals. The reflected light may be filtered through spatial filtering such as blocking of light around the center through absorption or an aperture, or the use of an annular detector.

Block 1124 may be followed by block 1126, “CAPTURE THE SPATIALLY FILTERED LASER LIGHT”, where the filtered light is captured by a detector to process the light and determine an effect of the laser treatment (e.g., formation of bubbles). In other examples, the entire reflected light may be captured by an array of detectors and a portion of the reflected light corresponding to a center area (within the pre-defined angle) may be filtered out through signal processing.

Block 1126 may be followed by optional block 1128, “PROCESS THE CAPTURED LASER LIGHT TO OBSERVE AN EFFECT OF THE DIRECTED LASER BEAM ON THE TREATMENT SITE”, where the captured light may be processed by deriving signal(s), processing the signals, and determining the effect of the directed laser beam in order to decide whether to cease the laser beam application or continue with further pulses, for example.

The operations included in process 1100 are for illustration purposes. Signal-to-noise ratio enhancement in reflectometry through capture of light scattered to the edge of the pupil and filtering of light scattered back from the pupil's center may be implemented by similar processes with fewer or additional steps, as well as in different order of operations using the principles described herein. The operations described herein may be executed by one or more processors operated on one or more computing devices, one or more processor cores, specialized processing devices, and/or general purpose processors, among other examples.

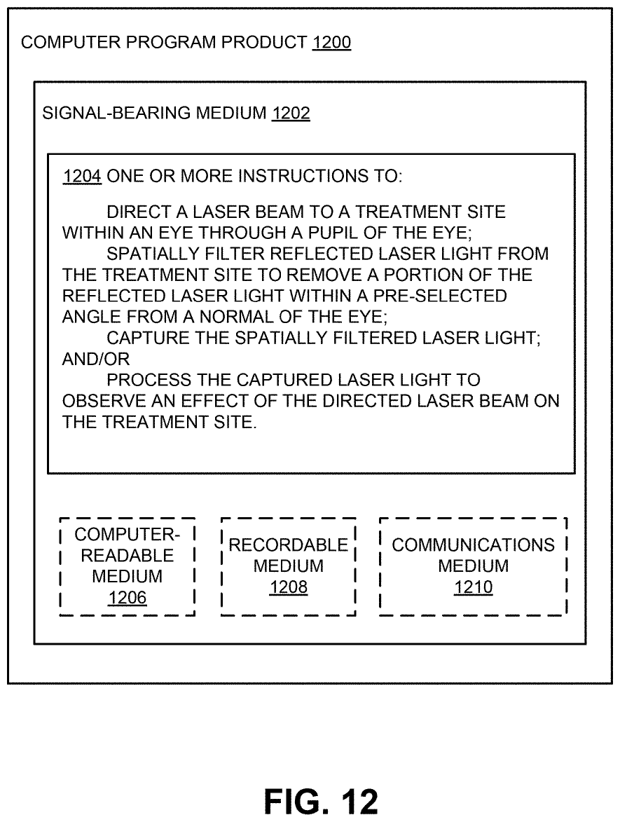

FIG. 12 illustrates a block diagram of an example computer program product, some of which are arranged in accordance with at least some embodiments described herein.

In some examples, as shown in FIG. 12, a computer program product 1200 may include a signal bearing medium 1202 that may also include one or more machine readable instructions 1204 that, when executed by, for example, a processor may provide the functionality described herein. Thus, for example, referring to the processor 1004 in FIG. 10, the laser control module 1022 may undertake one or more of the tasks shown in FIG. 12 in response to the instructions 1204 conveyed to the processor 1004 by the signal bearing medium 1202 to perform actions associated with the enhancement of signal-to-noise ratio in reflectometry through capture of light scattered to the edge of the pupil and filtering of light scattered back from the pupil's center as described herein. Some of those instructions may include, for example, direct a laser beam to a treatment site within an eye through a pupil of the eye; spatially filter reflected laser light from the treatment site to remove a portion of the reflected laser light within a pre-selected angle from a normal of the eye; and/or capture the spatially filtered laser light, according to some embodiments described herein.

In some implementations, the signal bearing medium 1202 depicted in FIG. 12 may encompass computer-readable medium 1206, such as, but not limited to, a hard disk drive (HDD), a solid state drive (SSD), a compact disc (CD), a digital versatile disk (DVD), a digital tape, memory, etc. In some implementations, the signal bearing medium 1202 may encompass recordable medium 1208, such as, but not limited to, memory, read/write (R/W) CDs, R/W DVDs, etc. In some implementations, the signal bearing medium 1202 may encompass communications medium 1210, such as, but not limited to, a digital and/or an analog communication medium (e.g., a fiber optic cable, a waveguide, a wired communication link, a wireless communication link, etc.). Thus, for example, the computer program product 1200 may be conveyed to one or more modules of the processor 1004 by an RF signal bearing medium, where the signal bearing medium 1202 is conveyed by the communications medium 1210 (e.g., a wireless communications medium conforming with the IEEE 802.11 standard).

According to some examples, a method to increase a signal-to-noise ratio of reflectometry in laser based eye treatment is described. The method may include directing a laser beam to a treatment site within an eye through a pupil of the eye; spatially filtering reflected laser light from the treatment site to remove a portion of the reflected laser light within a pre-selected angle from a normal of the eye; and capturing the spatially filtered laser light, where the signal-to-noise ratio of the reflectometry is increased through the spatial filtering.

According to other examples, the method may also include processing the captured laser light to observe an effect of the directed laser beam at the treatment site. Spatially filtering the reflected laser light from the treatment site may include determining the pre-selected angle from the normal of the eye based on a diameter of the laser beam. Spatially filtering the reflected laser light from the treatment site may also include removing the portion of the reflected laser light within about 1.2 degrees from the normal of the eye for a laser beam of about 100 μm diameter.

According to further examples, spatially filtering the reflected laser light from the treatment site may include removing another portion of the reflected laser light outside of about 9 degrees from the normal of the eye. Spatially filtering the reflected laser light from the treatment site may further include removing the portion of the reflected laser light through absorption or deflection. Spatially filtering the reflected laser light from the treatment site may also include offsetting a detector to capture the reflected laser light from the normal of the eye. Spatially filtering the reflected laser light from the treatment site may include employing a set of serially positioned detectors to capture the reflected laser light around the normal of the eye. Spatially filtering the reflected laser light from the treatment site may also include employing an aperture substantially centered in a detector to capture the reflected laser light.

According to other examples, another method to increase a signal-to-noise ratio of reflectometry in laser based eye treatment is described. The method may include directing a laser beam to a treatment site within an eye through a pupil of the eye; capturing reflected laser light from the treatment site through a plurality of detectors; deriving a plurality of signals from the captured laser light by the plurality of detectors; and removing a portion of the derived plurality of signals that corresponds to a portion of the captured laser light within a pre-selected angle from a normal of the eye to increase the signal-to-noise ratio of the reflectometry.

According to some examples, the method may further include processing a remaining portion of the plurality of signals to observe an effect of the directed laser beam at the treatment site. Removing the portion of the derived plurality of signals that corresponds to the portion of the captured laser light within the pre-selected angle from the normal of the eye to increase the signal-to-noise ratio of the reflectometry may include determining the pre-selected angle from the normal of the eye based on a diameter of the laser beam. Removing the portion of the derived plurality of signals that corresponds to the portion of the captured laser light within the pre-selected angle from the normal of the eye to increase the signal-to-noise ratio of the reflectometry further may also include removing the portion of the plurality of signals that correspond to the portion of the reflected laser light within about 1.2 degrees from the normal of the eye for a laser beam of about 100 μm diameter. Removing the portion of the derived plurality of signals that corresponds to the portion of the captured laser light within the pre-selected angle from the normal of the eye to increase the signal-to-noise ratio of the reflectometry may further include removing another portion of the plurality of signals that correspond to another portion of the reflected laser light outside of about 9 degrees from the normal of the eye.

According to further examples, a laser treatment system may include a laser configured to direct a laser beam to a treatment site within an eye through a pupil of the eye and a detection module coupled to the laser. The detection module may be configured to spatially filter reflected laser light from the treatment site to remove a portion of the reflected laser light within a pre-selected angle from a normal of the eye; and capture the spatially filtered laser light, where the signal-to-noise ratio of the reflectometry is increased through the spatial filtering.

According to other examples, the system may also include a processing module coupled to the laser and the detection module, the processing module configured to process the captured laser light to observe an effect of the directed laser beam at the treatment site. The system may further include a controller coupled to the laser, the detection module, and the processing module, the controller configured to control and coordinate operations of the laser, the detection module, and the processing module. To filter the reflected laser light from the treatment site, the detection module may be configured to determine the pre-selected angle from the normal of the eye based on a diameter of the laser beam.

According to yet other examples, to filter the reflected laser light from the treatment site, the detection module may be configured to remove the portion of the reflected laser light within about 1.2 degrees from the normal of the eye for a laser beam of about 100 μm diameter. To filter the reflected laser light from the treatment site, the detection module may be configured to remove another portion of the reflected laser light outside of about 9 degrees from the normal of the eye. The detection module may be configured to remove the portion of the reflected laser light through absorption or deflection. The detection module may also include an offset detector to capture the reflected laser light from the normal of the eye. The detection module may also include a set of serially positioned detectors to capture the reflected laser light around the normal of the eye. The detection module may further include an aperture substantially centered with a detector to capture the reflected laser light.

According to some examples, another laser treatment system may include a laser configured to direct a laser beam to a treatment site within an eye through a pupil of the eye; a detection module coupled to the laser and comprising a plurality of detectors, the detection module configured to capture reflected laser light from the treatment site through the plurality of detectors; and a processing module coupled to the laser and the detection module. The processing module may be configured to derive a plurality of signals from the captured laser light by the plurality of detectors; and remove a portion of the derived plurality of signals that corresponds to a portion of the captured laser light within a pre-selected angle from a normal of the eye to increase the signal-to-noise ratio of the reflectometry.

According to other examples, the processing module may be further configured to process a remaining portion of the plurality of signals to observe an effect of the directed laser beam at the treatment site. The processing module may also be configured to determine the pre-selected angle from the normal of the eye based on a diameter of the laser beam. The processing module may be further configured to remove the portion of the plurality of signals that correspond to the portion of the reflected laser light within about 1.2 degrees from the normal of the eye for a laser beam of about 100 μm diameter. The processing module may also be configured to remove another portion of the plurality of signals that correspond to another portion of the reflected laser light outside of about 9 degrees from the normal of the eye.

There is little distinction left between hardware and software implementations of aspects of systems; the use of hardware or software is generally (but not always, in that in certain contexts the choice between hardware and software may become significant) a design choice representing cost vs. efficiency tradeoffs. There are various vehicles by which processes and/or systems and/or other technologies described herein may be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for mainly hardware and/or firmware vehicle; if flexibility is paramount, the implementer may opt for mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples may be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, those skilled in the art will recognize that some aspects of the embodiments disclosed herein, in whole or in part, may be equivalently implemented in integrated circuits, as one or more computer programs executing on one or more computers (e.g., as one or more programs executing on one or more computer systems), as one or more programs executing on one or more processors (e.g., as one or more programs executing on one or more microprocessors), as firmware, or as virtually any combination thereof, and that designing the circuitry and/or writing the code for the software and/or firmware would be well within the skill of one of skill in the art in light of this disclosure.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also to be understood that the terminology used herein is for the purpose of describing particular embodiments only, and is not intended to be limiting.

In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive (HDD), a compact disc (CD), a digital versatile disk (DVD), a digital tape, a computer memory, a solid state drive (SSD), etc.; and a transmission type medium such as a digital and/or an analog communication medium (e.g., a fiber optic cable, a waveguide, a wired communication link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. Those having skill in the art will recognize that a data processing system may include one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors.

A data processing system may be implemented utilizing any suitable commercially available components, such as those found in data computing/communication and/or network computing/communication systems. The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no, such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method to increase a signal-to-noise ratio of reflectometry in laser-based eye treatment, the method comprising:
directing a laser beam to a treatment site within an eye through a pupil of the eye;
spatially filtering reflected laser light from the treatment site by removing a first portion of the reflected laser light within a pre-selected angle from a normal of the eye with a spatial filter; and
detecting a second portion of the reflected laser light that is passed through the spatial filter to a detector that provides a detected signal, wherein
the first and second portions of the reflected laser light are distinct, and
the spatial filtering is effective to increase a signal-to-noise ratio of the detected signal.

2. The method of claim 1, further comprising:
processing the detected signal to observe an effect of the directed laser beam at the treatment site.

3. The method of claim 1, wherein spatially filtering the reflected laser light from the treatment site comprises:
determining the pre-selected angle from the normal of the eye based on a spot size of the laser beam, wherein the pre-selected angle is in a range from about 1.2 degrees to about 2.0 degrees depending on the spot size of the laser beam.

4. (canceled)

5. The method of claim 1, wherein spatially filtering the reflected laser light from the treatment site comprises:
removing a third portion of the reflected laser light outside of another pre-selected angle in a range from about 5 degrees to about 9 degrees from the normal of the eye.

6. The method of claim 1, wherein spatially filtering the reflected laser light from the treatment site comprises one or more of:
removing the portion of the reflected laser light through absorption or deflection; or
offsetting the detector to capture the reflected laser light from the normal of the eye.

7. (canceled)

8. The method of claim 1, wherein spatially filtering the reflected laser light from the treatment site further comprises one or more of:
employing a set of serially positioned detectors to detect the second portion of the reflected laser light around the normal of the eye; or
employing an aperture substantially centered in the detector to detect the second portion of the reflected laser light.

9. (canceled)

10. A method to increase a signal-to-noise ratio of reflectometry in laser-based eye treatment, the method comprising:
directing a laser beam to a treatment site within an eye through a pupil of the eye;
detecting reflected laser light from the treatment site through a plurality of detectors to provide a detected plurality of signals from the detected laser light by the plurality of detectors; and
removing a first portion of the detected plurality of signals that corresponds to a first portion of the detected laser light within a pre-selected angle from a normal of the eye effective to increase a signal-to-noise ratio of a second portion of the detected plurality of signals that corresponds to a second portion of the detected laser light outside the pre-selected angle.

11. The method of claim 10, further comprising:
processing the second portion of the detected plurality of signals to observe an effect of the directed laser beam at the treatment site.

12. (canceled)

13. The method of claim 10, wherein removing the first portion of the detected plurality of signals that corresponds to the first portion of the captured laser light within the pre-selected angle from the normal of the eye further comprises one or more of:
removing the first portion of the detected plurality of signals that correspond to the first portion of the reflected laser light within a range of the pre-selected angle from about 1.2 degrees to about 2.0 degrees from the normal of the eye depending on a spot size of the laser beam; or
removing a third portion of the detected plurality of signals that correspond to a third portion of the reflected laser light outside of another pre-selected angle in a range from about 5 degrees to about 9 degrees from the normal of the eye.

14. (canceled)

15. A laser treatment system comprising:
a laser configured to direct a laser beam to a treatment site within an eye through a pupil of the eye;
a detection module coupled to the laser and configured to:
spatially filter reflected laser light from the treatment site to remove a first portion of the reflected laser light within a pre-selected angle from a normal of the eye through absorption or deflection; and
detect a second portion of the reflected laser light that is spatially filtered at the detector module to provide a detected signal, wherein
the first and second portions of the reflected laser light are distinct, and
the spatial filtering is effective to increase a signal-to-noise ratio of the detected signal.

16. The system of claim 15, further comprising:
a processing module coupled to the laser and the detection module, the processing module configured to process the detected signal to observe an effect of the directed laser beam at the treatment site.

17. The system of claim 16, further comprising:
a controller coupled to the laser, the detection module, and the processing module, the controller configured to control and coordinate operations of the laser, the detection module, and the processing module.

18. The system of claim 15, wherein, to filter the reflected laser light from the treatment site, the detection module is configured to:
determine the pre-selected angle from the normal of the eye based on a spot size of the laser beam, wherein the pre-selected angle is in a range from about 1.2 degrees to about 2.0 degrees depending on the spot size of the laser beam.

19. (canceled)

20. The system of claim 15, wherein, to filter the reflected laser light from the treatment site, the detection module is configured to:
remove a third portion of the reflected laser light outside of another pre-selected angle in a range from about 5 degrees to about 9 degrees from the normal of the eye.

21. (canceled)

22. The system of claim 15, wherein the detection module comprises one or more of:
an offset detector to detect the reflected laser light from the normal of the eye; or
a set of serially positioned detectors to detect the reflected laser light around the normal of the eye.

23. (canceled)

24. The system of claim 15, wherein the detection module comprises:
an aperture substantially centered with a detector to detect the reflected laser light.

25. A laser treatment system comprising:
a laser configured to direct a laser beam to a treatment site within an eye through a pupil of the eye;
a detection module coupled to the laser and comprising a plurality of detectors, the detection module configured to detect reflected laser light from the treatment site through the plurality of detectors to provide a detected plurality of signals from the detected laser light; and
a processing module coupled to the laser and the detection module, the processing module configured to:
receive the detected plurality of signals from the detection module; and
remove a first portion of the detected plurality of signals that corresponds to a first portion of the detected laser light within a pre-selected angle from a normal of the eye effective to increase the signal-to-noise ratio of a second portion of the detected plurality of signals that corresponds to a second portion of the detected laser light outside the pre-selected angle.

26. The system of claim 25, wherein the processing module is further configured to:
process the second portion of the detected plurality of signals to observe an effect of the directed laser beam at the treatment site.

27. (canceled)

28. The system of claim 25, wherein the processing module is further configured to:
remove the first portion of the detected plurality of signals that correspond to the first portion of the reflected laser light within a range of the pre-selected angle from about 1.2 degrees to about 2.0 degrees from the normal of the eye depending on a spot size of the laser beam.

29. The system of claim 25, wherein the processing module is further configured to:
remove a third portion of the detected plurality of signals that correspond to a third portion of the reflected laser light outside of another pre-selected angle in a range from about 5 degrees to about 9 degrees from the normal of the eye.
